(19) United States
(12) Patent Application Publication
(10) Pub. No.: US 2016/0095801 A1
(43) Pub. Date: Apr. 7, 2016

(54) MULTI-COMPONENT DENTAL WHITENING COMPOSITIONS

(71) Applicant: Pac-Dent International, Inc., Brea, CA (US)

(72) Inventors: Susan Jiao, Irvine, CA (US); Daniel Wang, Rowland Heights, CA (US); Bo Tao, Chino, CA (US)

(73) Assignee: Pac-Dent International, Inc., Brea, CA (US)

(21) Appl. No.: 14/959,991

(22) Filed: Dec. 4, 2015

Related U.S. Application Data
(60) Provisional application No. 62/060,169, filed on Oct. 6, 2014.

(51) Int. Cl. A61K 8/19; A61K 8/22; A61K 8/44; A61K 8/34; A61K 8/25; A61Q 11/00; A61K 8/86

(52) U.S. Cl. CPC A61K 8/19; A61Q 11/00; A61K 8/22; A61K 8/86; A61K 8/345; A61K 8/25; A61K 8/44; A61K 2800/882; A61K 2800/87; A61K 2800/592; A61K 2800/58

(57) ABSTRACT

A two-component tooth whitening system containing a bleaching gel and an activating gel, including that the system has both thermo- and foaming-stability for non-refrigerated storage. The admixing of the two components provides effective whitening and proper bubbling that enables oxygen and free radicals released to diffuse into tooth enamel, and thus, provides significant whitening efficacy.

### CROSS REFERENCE TO RELATED APPLICATION

This application claims benefit under 35 U.S.C. §119(e) to U.S. Provisional Application No. 62/060,169, filed Oct. 6, 2014, which is incorporated by reference herein in its entirety.

### BACKGROUND OF THE INVENTION

1. Field of the Invention

The present invention generally relates to a multi-component tooth whitening system having both thermo and foaming stability for non-refrigeration storage, which gel provides effective whitening once the two components are mixed and applied on tooth surface.

2. Background Information

The importance of tooth whitening for patients and consumers has seen a dramatic increase in the number of products and procedures over recent years. There are usually two forms of tooth whitening: in-office and at-home bleaching. The in-office bleaching utilizes a high concentration of peroxide bleaching agent, with peroxide concentrations making up to 35% of the whitening formulation. Typically, the whitening formulation is applied to the teeth after protection of the soft tissue. In-office bleaching compositions are usually categorized as being either a “one-part” or “two-part” system. In the “two-part” system the components which would otherwise interact with the peroxide-bleaching agent are physically separated from one another prior to use.

High concentrations of hydrogen peroxide in the bleaching gel of the two-part system benefits whitening efficacy, but is known to cause storage difficulties: e.g., hydrogen peroxide is prone to decompose, where the gel can lose viscosity and liquefy, leading leak out or blow out of the dispenser. Several approaches have been tried to overcome these difficulties, while at the same time increasing the storage stability of the bleaching gel. For example, U.S. Pat. No. 5,059,417 has focused on maximizing the stability via incorporating glycerol and polyoxyethylene-polyoxypropylene copolymer in a ratio ranging from 1:0.7 to 1:0.4 and modulating acidifying pH to less than 3. However, the acid pH of the bleaching gel can cause demineralization of the teeth, and thus, tooth discomfort, especially for patients with “sensitive” teeth. In another example, U.S. Pat. No. 6,485,709 claims a dental bleaching gel having a shelf life as long as 6 months, comprising hydrophilic copolymer of polyethylene oxide, including polypropylene oxide as a thickening agent, and at least one bleaching agent stabilizer for minimizing the effect of a peroxide agent on dye discoloration during storage. Further, U.S. Pat. No. 6,555,020 discloses that the combination of polyacrylic acid thickener and aminocarboxylic acid/salt stabilizer that may be used to make a gel that is stable for 4-12 weeks at room temperature.

In view of all of the aforementioned art, it is apparent that hydrogen peroxide compositions may be stabilized by using bleaching agent-stabilizers and stable thickening-agents, however, typically, the shelf life of the resulting gels is limited to 6 months at room temperature. To increase the stability of dental bleaching gels containing high concentrations of hydrogen peroxide, the bleaching gels are generally refrigerated prior to use. And current whitening products require refrigeration during transportation and storage, which increases the cost and results in other inconveniences for users.

The objective of the present invention is to provide a bleaching gel that is stable against both heat and potential foaming, and thus, does not require refrigeration storage.

The art recognized mechanism of tooth whitening by peroxide occurs by the diffusion of peroxide through enamel to produce free radicals that oxidize colored organic molecules and inorganic compounds. The mechanisms of these reactions are varied and dependent on the substrate, the reaction environment, and catalysis. In general, these reactions may form a number of different active oxygen species depending on reaction conditions, including, but not limited to temperature, pH, light and presence of transition metals. In order to accelerate the releasing of free radicals, professionals have used devices that transfer energy to hydrogen peroxide, increasing its decomposition. In general, the activating sources used are the halogen lamp, light emitting diode (LED), plasma arc, and laser. However, light application may cause a temperature rise and increase the diffusion of the bleaching agent through the dental hard tissues, eventually reaching the pulpal chamber.

Light activation leads to gel heating. When the temperature rises, the collisions among the gel molecules turn out to be more frequent, increasing the likelihood of bond cleavage and other reactions, while at the same time accelerating diffusion speed. However, in vivo studies comparing various tooth bleaching therapies, with and without light activation, are controversial with respect to the real contribution of light irradiation to the final outcome of dental bleaching modalities. For example, Dias Ribeiro et al. (Ribeiro et al., Cytotoxic effect of a 35% hydrogen peroxide bleaching gel on odontoblast-like MDPC-23 cells. Oral Surg Oral Med Oral Pathol Oral Radiol Endod (2009) 108:458-464) found that 35% hydrogen peroxide (HP) bleaching gel associated with light activation presented transenamel and transdentinal cytotoxic effects characterized by direct damage to odontoblasts and a decrease of their metabolic activity. For these authors, from a biological point of view, the application of light or heat on the bleaching gel to catalyze HP degradation and speed up tooth bleaching is questionable.

As an attempt to increase the efficacy and maintain the safety of the treatment, chemical activating agents have been incorporated in the bleaching gels. There are usually three types of activating agents: high pH activator containing alkali such as potassium hydroxide, sodium carbonate or bicarbonate; enzyme and metal ions. Each activating agent may accelerate the decomposition of hydrogen peroxide in its own unique way. For example, under alkaline conditions, hydrogen peroxide bleaching generally proceeds via the perhydroxyl anion (HO₂⁻). U.S. Pat. No. 5,928,628 claims a component having pH from 9-13 and pH of the admix from 8.5 to 11 to thereby increase the rate of active oxygen and accelerate the bleaching action. However, a high pH would be toxic and irritant to the oral cavity. U.S. Pat. No. 6,485,709 claims an activator system, including dried particles of a soluble activator comprising enzyme catalase, for accelerating the bleaching action. Using catalase, an organo-metallic enzyme containing iron, a very large amount of hydroxyl free radicals, and hence a large amount of oxygen, would be released in a short time, and would in turn may provide good bleaching action. However, the destabilizing action provided by catalase may not necessarily provide a concomitant extensive bleaching action due to a different mechanism of destabilizing. Plus, the enzyme is not stable to high temperature storage, and may lose its activating efficacy prior to use.

Transition metals can react with hydrogen peroxide, accelerating the production of free radicals and thus obtaining a faster, more efficient, and safer bleaching process, while at the same time reducing the potential damaging effects associated with the application of heat sources. In addition, there may be some influence on the penetration of hydrogen peroxide, since a reaction is expected to occur between the hydrogen peroxide and the chemical activator. Some research papers (e.g., Travassos et al., In Vitro Assessment of Chemical Activation Efficiency During In-office Dental Bleaching, Operative Dentistry (2010) Vol. 35, No. 3: 287-294) demonstrate that when manganese gluconate was added to the bleaching gel, the metal's salts decompose hydrogen peroxide and form free radicals that react with the tooth structure and hence the whitening efficacy is improved. U.S. Pub. No. 2004/0191188 (provisional application No. 60/459,183) claims a composition for boosting and activating tooth-whitening agents, in particular hydrogen peroxide. The composition includes a heavy-metal salt solution, and a chelator for preventing the heavy metal salt solution from precipitating, adjusted to a basic pH, preferably above 10.5. This alkaline pH could increase hydrogen peroxide decomposition and thus accelerate its bleaching action. However, high pH would be toxic and irritant to the oral cavity. Plus, at high pH, the metal-chelator complex is unstable and prone to be oxidized to colored compounds prior to use, and thus becomes ineffective.

The objective of the present invention is to provide a stable activating gel for room temperature storage. The activating gel comprises of stabilized manganese complex to accelerate the bleaching action of hydrogen peroxide, and has slightly alkaline pH from 7.5 to 8.9, to ensure safe but effective whitening.

### SUMMARY OF THE INVENTION

The present invention is directed to a multi-component dental bleaching system, which is stable to both heat and foaming for non-refrigeration storage. The whitening system of the present invention comprises of a bleaching gel and an activating gel. The bleaching gel composition comprises hydrogen peroxide as bleaching agent, at least one bleaching agent stabilizer and a stable thickening agent to form viscous gel, and more particularly, at least one foam stabilizer that minimizes the potential foaming of hydrogen peroxide during transportation and storage. The activating gel of the present invention has slightly alkaline pH from 7.5 to 8.9. The activating gel comprises a transition metal complex to activate the bleaching action of hydrogen peroxide, and more particularly, at least one complex stabilizer for further stabilizing the metal complex to improve diffusion of the active oxygen and free radicals generated by hydrogen peroxide decomposition.

The admixing of the bleaching gel and activating gel has a neutral pH from about 7.5 to 8.0. It provides effective whitening and controlled bubbling that enables oxygen and free radicals released to diffuse into tooth enamel, and thus results in significant whitening efficacy.

In embodiments, a multi-component dental bleaching system is disclosed including a dental bleaching composition comprising a bleaching gel and an activator gel, where the bleaching gel comprises at least one peroxide, at least one bleaching agent stabilizer, at least one thickening agent and at least one foam stabilizer, and where the activator gel comprises a transition metal complex, at least complex stabilizer, and optionally a flavorant.

In one aspect, the at least one peroxide includes hydrogen peroxide, carbamide peroxide, urea peroxide, perborate, peroxyacetic acid, and combinations thereof In another aspect, the at least one bleaching agent stabilizer includes a chelator, a tin salt, an alkali stannate, a phosphate, a pyrophosphate, a phosphoric acid, and combinations thereof

In one aspect, the at least one thickening agent is selected from the group consisting of poly(ethylene oxide) (PEO), polyvinyl oxide (PVP), a copolymer of PEO and polypropylene oxide (PPO). In another aspect, the bleaching agent further contains a humectant including glycerin, liquid polyethylene glycol (PEG), liquid polypropylene glycol, propylene glycol and combinations thereof In a further aspect, the foam stabilizer includes at least one non-ionic surfactant and at least on colloidal silica.

In another aspect, the transition metal complex is formed by complexation of transition metal ions with a chealating agent, wherein the transition metal ions are selected from soluble manganese, cobalt, nickel, and copper salts and the chelating agent is including EDTA and its salts such as disodium EDTA salt, trisodium EDTA salt and tetrasodium EDTA salt and mixture thereof In further aspect, the complex stabilizer is selected from a group of non-ionic polymer surfactants such as polyvinyl alcohol, polyoxyethylene-polypropylene block copolymer, PVP, or PEG.

In one aspect, the complex comprises transition metal-gluconate salt. In another aspect, the non-ionic polymer surfactant is selected from polyvinyl alcohol, polyoxyethylene-polypropylene block copolymer, PVP, or PEG. In another aspect, the dental bleaching system is in the form of a kit that includes a bleaching gel in a first part and an activator gel in a second part. In a related aspect, the kit includes at least one syringe containing the bleaching gel and at least one additional syringe containing the activator gel. In a further related aspect, the kit includes at least one duel compartment syringe containing a bleaching gel in a first compartment and an activator gel in a second compartment.

In one aspect, the bleaching gel is mixed with the activator gel in a ratio from about 1:1 to about 5:1. In another aspect, the bleaching system is stable in consistency and activating efficacy for up to 12 months at room temperature. In a related aspect, the activator gel has a pH of from about 7.5 to about 8.9, and the bleaching system has a final pH of about 7.0 to about 8.5. In a further related aspect, the bleaching system comprises a flavorant and at least one humectant.

In another embodiment, a method of bleaching a subject's teeth is disclosed including applying a multi-component dental bleaching system containing a dental bleaching composition comprising a bleaching gel and an activator gel, where the bleaching gel comprises at least one peroxide, at least one bleaching agent stabilizer, at least one thickening agent and at least one foam stabilizer, and where the activator gel comprises a transition metal complex and at least one complex stabilizer; mixing the bleaching gel and the activator gel in a ratio from about 1:1 to 5:1; and applying the mixed system to at least a portion of the subject's teeth.

In another aspect, the bleaching system is contained in a kit comprising at least one duel compartment syringe containing the bleaching gel in a first compartment and the activator gel in a second compartment. In a related aspect, the activator gel has a pH of from about 7.5 to about 8.9, and the bleaching system has a final pH of about 7.0 to about 8.5. In a further related aspect, the bleaching system comprises a flavorant and a humectant.

### DETAILED DESCRIPTION OF THE INVENTION

Before the present composition, methods, and methodologies are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “a chelator” includes one or more chelators, and/or compositions of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Any methods and materials similar or equivalent to those described herein can be used in the practice or testing of the invention, as it will be understood that modifications and variations are encompassed within the spirit and scope of the instant disclosure.

As used herein, “about,” “approximately,” “substantially” and “significantly” will be understood by a person of ordinary skill in the art and will vary in some extent depending on the context in which they are used. If there are uses of the term which are not clear to persons of ordinary skill in the art given the context in which it is used, “about” and “approximately” will mean plus or minus <10% of particular term and “substantially” and “significantly” will mean plus or minus >10% of the particular term. In embodiments, composition may “contain”, “comprise” or “consist essentially of” a particular component of group of components, where the skilled artisan would understand the latter to mean the scope of the claim is limited to the specified materials or steps “and those that do not materially affect the basic and novel characteristic(s)” of the claimed invention.

As used herein, “multi-component” means more than one part. For example, the bleaching dental system as disclosed herein comprises a bleaching gel and an activator gel, which are separate, and are admixed to achieve the bleaching effect of the instant invention.

As used herein, “activator efficiency (AE)” means the time consumed for color of the activating gel mixed with a control bleaching gel and dye solution to fade (i.e., turn colorless).

As used herein, “bleaching efficiency (BE) ” means the time consumed for the color of the bleaching gel mixed with a control activating gel and a dye solution to fade (i.e., turn to colorless).

As used herein, “gel” means a colloid in a more solid form than a sol.

As used herein, “stable in consistency” means that the gel state of matter does not significantly change overtime and remains in said gel state relative to time 0. For example, gels may be considered stable if a gel droplet remains in place for a 5 minute time period without cascading down a glass slide that is placed vertically.

As used herein, “complexation”, including grammatical variations thereof, means the combination of individual atom groups, ions or molecules to create one large ion or molecule.

It has been found that there are several critical conditions required to achieve a chemically stable bleaching gel. These conditions required include bleaching agent stabilizers, acidic pH level, and stable thickening agent. One previous patent (U.S. Pat. No. 6,485,709) provided a bleaching composition having a six-month shelf life at room temperature and a shelf life at 4° C. in a regular refrigerator for at least 12 months. In embodiments, for the instant system, to achieve a long shelf life at room temperature for a non-refrigerated storage, at least one foam stabilizer may be added to form the bleaching gel.

There are three classes of stabilizers for stabilizing hydrogen peroxide. Some stabilizers, such as disodium EDTA, act as metal scavengers since metal ions can react as reductant with hydrogen peroxide and thus activate peroxide decomposition. Some stabilizers, such as sodium sulfite, act as antioxidants. The other stabilizers such as tin salts, alkali stannate, phosphates, pyrophosphates, phosphoric acid, and the like may specifically stabilize hydrogen peroxide. The stabilizer used in the present invention is selected from the abovementioned compounds and mixtures thereof In embodiments, the foam stabilizer may be present from about 0.1% to about 10%, from about 0.1% to about 20%, and about 0.1% to about 35%. In embodiments, the foam stabilizer may be present from about 0.5% to about 15% by weight of the final composition.

While not being bound by theory, the stability of hydrogen peroxide seems to decrease with increasing alkalinity. Therefore, the pH of the bleaching gel may be kept below 7. However, the gel would etch the enamel and demineralize the teeth at acidic pH. The pH level of the bleaching gel of the present invention is below 7, preferably, between about 4 to about 6.

Thickening agents should be used to obtain a viscous gel so that it can cling to the surface of the teeth during whitening treatment. Due to the strong oxidizing ability of hydrogen peroxide, the thickening agent used should be stable or inert to hydrogen peroxide in the bleaching composition. In view of the stability or gelling ability at acidic pH, the thickening agent should be stable or at least should be efficacious at acidic pH. When cross-linked polyacrylic acid was added as thickening agent, the bleaching gel turned to liquid after seven days of incubation at 50° C., while other batches prepared with the stable thickening agent kept on gel form even after 1.5 month incubation at 50° C. Therefore, the cross-linked polyacrylic acid is not considered as stable thickening agent. A preferred thickening agent is poly(ethylene oxide) with molecular weight more than 10,000, polyvinylpyrrolidone (PVP), copolymer of polyethylene oxide and polypropylene oxide, and mixtures of thereof. To form a viscous gel and maintain the moisture of the gel during storage, some humectant may be added. In the present invention, the preferred humectant includes glycerin, liquid polyethylene glycol, liquid polypropylene glycol, propylene glycol, and others. The humectant components may be used singly or in combination. In some embodiments, a mixture of propylene glycol/glycerin may be used as humectants. The amount of the humectants should be from about 5% to about 30%.

In addition, at higher temperatures (23° C./73° F. or higher), hydrogen peroxide tends to continuously decompose and release oxygen, resulting in pressure buildup inside its container, which may eventually lead to explosion. Therefore, the presence of one or more foam stabilizers in the bleaching gel formulation is particularly critical to the stability for long term non-refrigeration storage. In the present invention, the foam stabilizer may be a mixture of non-ionic surfactants and colloidal silica. The colloidal silica particles are initially hydrophilic. The surfactants that have hydrophilic polyethylene oxide chain (C₂H₄O)n, similar to poly(ethylene) oxide (PEG) or Triton (C₁₄H₂₂O(C₂H₄O)n may be added into the silica suspension at certain pHs. The hydrophobicity of the silica nano-particles increases significantly because of the adsorption of the surfactants by the particle surfaces. As the hydrophobicity of the particles becomes the main factor for stabilization mechanism (mainly due to their firm attachment on the gas-liquid/air-water interfaces), a sufficient concentration of nano-particles is considered as an influenced parameter rising from their saturation inside the thinning film which will further slow down the liquid drainage by forming steric barrier against coalescence. Experiments indicated that the silica/surfactant mixture was indeed effective in stabilizing foam. Moreover, it was found that the synergetic effect of silica/surfactant in stabilizing the foam occurs mainly at low and intermediate surfactant concentration. At intermediate surfactant concentration (0.1%), foams are much more stable than at higher silica concentration. In the present invention, the foam stabilizer mixture comprises from about 0.5% to about 15% by weight of the total composition. In addition, when the bleaching gel is mixed with the activating gel containing transition metal complex, the metal's salts decompose hydrogen peroxide and a considerable amount of oxygen bubbles are formed. The formed oxygen bubbles remained homogeneous and controllable with the presence of the foam stabilizer, resulting in continuous diffusion of the active oxygen and free radicals, thus achieving much more effective whitening results.

In embodiments, the ingredients of the bleaching gel may be mixed under vigorous agitation until the powdered ingredients are fully dissolved or well dispersed, and a homogenous gel is obtained. For example, gel compositions may contain the components as shows in Table 1.

TABLE 1
Bleaching Gel Compositions.

| Bleaching Gel | Weight Percent % | | |
|---|---|---|---|
| Ingredients | Gel 1 | Gel 2 | Gel 3 |
| Water (De-ionized) | 6 | 2 | 0 |
| Hydrogen Peroxide | 40 | 60 | 80 |
| (50% solution) | | | |
| Disodium EDTA | 0.3 | 0.3 | 0.5 |
| Nano Silica (200 nm) | 6 | 6 | 8 |
| PEG 4000 | 0.7 | 2 | 3 |
| PEO-PPO co-polymer | 30 | 20 | 3 |
| Glycerin | 17 | 9.7 | 5.5 |

In embodiments, an activator gel is provided as a second component of a dual-barrel system as disclosed herein. Composition ranges of components in the activator gel may be found in Table 2.

TABLE 2
Composition Ranges of Various Ingredients
used in the Activator Gel.

| Ingredient | Range (%) |
|---|---|
| De-ionized water | 40-60 |
| Manganese gluconate | 0.1-2.9 |
| Chelator | 2-10 |
| Polymeric surfactant | 0.5-15 |
| Thickening agent | 1-8 |
| Desensitizers | 5-10 |
| pH adjustor | 1-5 |
| Buffering agent | 1-15 |
| Anti-calculus agent | 0.3-1 |
| Humectants | 5-30 |
| Flavorant | 0.1-1 |

In embodiments, the activating gel contains transition metal salts, for example, present at about 0.1% to about 2.9% manganese salts. In order to reduce demineralization caused by acidic or toxicity due to the alkaline pH of the admixing of the bleaching and activating gels, the activating gel should have alkaline pH or at least a neutral pH, since the bleaching gel has pH less than about 7. In embodiments, the activator gel may have a slightly alkaline pH from about 7.5 to about 8.9, and the mixture of the bleaching gel and the activator gel may have a neutral pH from about 7.0 to about 8.5. The pH of the activating gel may be adjusted with a pH adjusting agent. In a related aspect, pH-adjusting agents include, but are not limited to, potassium hydroxide, sodium hydroxide, ammonium hydroxide, tris(hydroxymethyl)aminomethane, triethanolamines, and the like. However, at high pH level (i.e., 7 and above), manganese salt may precipitate and gradually turn brown due to forming hydrated manganese (III) oxide, which diminishes its usefulness and results in a lower efficacy in activating hydrogen peroxide decomposition. While not being bound by theory, this might be the reason that higher concentrations of manganese salts were used in the previous patent/applications. For example, 5-10% of manganese citrate solution in water was added in U.S. Pat. No. 6,485,709; and 3% of manganese gluconate was used in a booster and activator composition in U.S. Provisional App. No. 60/459183 or U.S. Pub. No. 2004/0191188.

In embodiments, in order to prevent precipitation and keep higher activating efficacy, a chelating agent may be added to complex the manganese compound. In a related aspect, the chelating agent includes EDTA and its salts such as tetrasodium EDTA salt, trisodium EDTA salt, disodium EDTA salt and mixtures thereof

In embodiments, in order to further stabilize the manganese complex, a complex stabilizer comprising polymeric surfactants may be added. The polymeric surfactants may homogeneously disperse the manganese complex by adsorption of ligand, and thus prevent aggregation or precipitation and maintain the activating efficacy. The polymeric surfactants should have high binding affinities to the manganese complex. In embodiments, the polymeric surfactants include polyethylene glycol with molecular weight greater than 2000, polyoxyethylene/polyoxypropylene block copolymer, polyvinylpyrrolidone (PVP), polyvinyl alcohol and the like. Moreover, these polymeric surfactants may also improve the diffusion of the free radicals in the admixing gel, and thus achieve effective whitening results during whitening treatment. The activating gel may further comprise a bioadhesive polymer, the cross-linked polyacrylic acid, for example, from about 1% to about 8%. In addition, the activator gel may include a desensitizer in order to reduce tooth sensitivity and improve comfortability of the bleaching procedure.

In embodiments, the desensitizer may be potassium nitrate present at about 5% to about 10% by weight. In other embodiments of the activator gels, calcium hydroxyapatite in an amount of about 2% to about 5% by weight may be added to occlude open tubules and form a protective new layer made of tooth enamel, and thus eliminate sensitivity. To provide a pleasant flavor and color distinction upon whitening treatment, flavoring such as peppermint oil with an amount of up to about 1% by weight and a colorant such as FD & C Blue #1, FD & C Red #33 and the like may be used. In one aspect, an anti-calculus agent such as water-soluble alkali metal salt of polyphosphate may be added to protect the teeth against plaque. Sodium fluoride may be added to provide anti-cavity properties.

In embodiments, preparations of an activator gel may contain manganese gluconate, disodium EDTA, sodium fluoride, potassium nitrate, sodium citrate, sodium polyphosphate, which components may be dissolved in de-ionized water and then a cross-linked polyacrylic acid may be added under vigorous agitation until the powder is fully dissolved or is well dispersed. A pH adjustor, such as potassium hydroxide, sodium hydroxide, ammonium hydroxide, and the like, may be added slowly and mixed well. Humectants, such as glycerin, propylene glycol and flavorant may also be optionally added. A non-limiting example of activator gels may be found in Table 3:

TABLE 3
Activator Gel.

| Activator Gel | Weight Percentages (%) | | |
|---|---|---|---|
| Ingredients | Gel 4 | Gel 5 | Gel 6 |
| De-ionized water | 50 | 50 | 50 |
| Manganese gluconate | 0.8 | 1.2 | 1.5 |
| Disodium EDTA | 3 | 4 | 5 |
| PEG 4000 | 5 | 8 | 10 |
| Sodium polyphosphate | 1 | 1 | 1 |
| Potassium nitrate | 5 | 5 | 5 |
| Sodium citrate | 10 | 10 | 10 |
| Sodium fluoride | 0.555 | 0.555 | 0.555 |
| Polyacrylic acid | 5 | 5 | 5 |
| Sodium hydroxide | 1.6 | 1.6 | 1.6 |
| Glycerin | 17 | 12.6 | 9.3 |
| Flavorant | 1 | 1 | 1 |
| QS | 0.045 | 0.045 | 0.045 |

The tooth whitening compositions of the present invention comprises a two-component system presented in dual-barrel syringes. In embodiments, an active bleaching agent, a peroxide, may be present at above about 15% by weight, preferably from about 15% by weight to about 40% by weight peroxide. In one aspect, the peroxide includes hydrogen peroxide, carbamide peroxide, urea peroxide, perborate, peroxyacetic acid, and combinations thereof In embodiments, the peroxide is hydrogen peroxide.

In embodiments, a two-component thermo-stable and foam stable whitening system is disclosed including a bleaching gel as a first component and an activator gel as a second component, while the bleaching gel is mixed with the activator gel in a ratio from 1:1 to 5:1. In one aspect, the first component comprises essentially about 10-40% by weight hydrogen peroxide stabilized with at least one bleaching stabilizer, and more particularly, at least one foam stabilizer for minimizing hydrogen peroxide foaming during storage, and 0.1-35% by weight a stable thickening agent to form a viscous gel. In a related aspect, a foam stabilizer containing in the bleaching gel as disclosed is a mixture of colloidal silica and a polymeric surfactant. In another related aspect, the bleaching gel is stable to both heat and foaming for up to 12 months at room temperature. In a further related aspect, the activating gel is stable in consistency and activating efficacy for up to 12 months at room temperature.

The foam stabilizer, may include at least one polymeric surfactant having hydrophilic polyethylene oxide chain (C₂H₄O)n, such as polyethylene glycol with molecular weight more than 600 Da, Triton (C₁₄H₂₂O(C₂H₄O)n) and the like. Further, the foam stabilizer may contain from about 0.5% to about 15% by weight of the total composition weight.

In embodiments, the second component may consist essentially of a transition metal complex for activating the decomposition of hydrogen peroxide and at least one non-ionic polymeric surfactant for further stabilizing the metal complex and to accelerate diffusion of the released oxygen and free radicals into the tooth enamel.

In embodiments, a non-ionic polymeric surfactant may be present in the activator gel, where the surfactant acts as a transition metal complex stabilizer since it has high binding affinity to the transition metal complex and thus may disperse the complex and protect it against aggregation. In a related aspect, the non-ionic polymeric surfactant may include, but is not limited to, polyvinyl alcohol, polyoxyethylene/polyoxypropylene block copolymer, polyvinylpyrrolidone (PVP), polyethylene glycol with molecular weight greater than 2000 Da, and the like. The non-ionic polymeric surfactant may be present at about 0.5% to about 15% by weight of the total composition weight.

In embodiments, the bleaching agent stabilizer includes, but is not limited to, EDTA and its salt, potassium stannate, sodium stannate, etidronic acid, phosphoric acid, sodium pyrophosphate. In other embodiments, the stable thickening agent includes, but is not limited to, poly(ethylene) oxide with molecular weight greater than about 10,000 Da, polyvinylpyrrolidone (PVP), copolymer of polyethylene oxide and polypropylene oxide, and mixtures thereof. In a related aspect, the stable thickener comprises of about 0.1% to about 35% by weight of the total composition weight.

In embodiments, the system as disclosed herein comprises of a mixture of propylene glycol/glycerin as humectant from about 5% to about 30%, to form a homogeneous gel with proper viscosity that may remain on teeth during whitening.

In embodiments, in the system as disclosed the activator gel has a slightly alkaline pH from about 7.5 to about 8.9, and hence, the mixture of the bleaching gel and the activator gel has a neutral pH from about 7.0 to about 8.5. In a related aspect, the pH may be adjusted with a pH adjusting agent including, but not limited to, sodium hydroxide, potassium hydroxide, ammonium hydroxide, triethynolamine, tris(hydroxymethyl)aminomethane. In a related aspect, the transition metal of the activator gel contains a complex comprising about 0.1% to about 2.9% manganese complexed with a chelator containing EDTA salt. In a further related aspect, the chelator may be a water soluble EDTA salt, including, but not limited to, tetrasodium EDTA salt, trisodium EDTA salt and disodium EDTA salt. In a further related aspect, the activating gel may contain a bioadhesive polymer, i.e., the cross-linked polyacrylic acid, from about 1% to about 6%.

In embodiments, the dental bleaching system as disclosed herein may be contained in a kit, where the kit comprises a bleaching gel, an activating gel, a container, a label, an applicator (e.g., two compartment syringe, trays, separate containers and the like which would be apparent to the skilled artisan), instructions, including how to access instructions on the world wide web.

Unless otherwise indicated, % is by weight/weight percent. The following examples are intended to illustrate but not limit the invention.

### EXAMPLES

Example 1 and Example 2 are exemplary bleaching gels.

In order to predict the shelf life of the bleaching gel, accelerated stability studies were performed at accelerated conditions. Briefly, the gel was filled in 1.2 mL syringes and then placed in the oven set to 50° C. and 37° C. At a predetermined time point the syringes were taken out and the formulations were evaluated in terms of hydrogen peroxide (HP) concentration or percentage retained, gel consistency, and whitening efficacy (in vitro on a FD & C dye). A long-term stability study was also undertaken at room temperature (23° C.). HP concentration was titrated by potassium permanganate assay (U.S. Pharmacopeia, USPXXII NFXVII, 1995, pp. 767). The gel consistency was evaluated by extruding a droplet of the gel onto a glass slide and placing the slide in a vertical position. Gels were considered acceptable if the gel droplet remain in place for a 5 minute time period without cascading down the glass slide that was placed vertically. The bleaching efficacy (BE) was assessed by the time consumed for the color of the bleaching gel mixed with the control activating gel and a dye solution to apparently fade (turn to colorless), so, the shorter the time, the stronger the bleaching efficacy of the bleaching gel. Note that the result is not considered as significantly different if the time difference is within 5-20 min.

Table 4 shows the desired ranges for the constituents of the bleaching gel. All the ingredients were mixed until the powdered ingredients were fully dissolved or well dispersed and a homogenous gel was obtained.

TABLE 4
Bleaching Gel Compositions.
COMPOSITION RANGES OF VARIOUS
INGREDIENTS USED IN THE BLEACHING GEL

| INGREDIENT | RANGE (%) |
|---|---|
| HYDROGEN PEROXIDE | 20-80 |
| BLEACHING AGENT STABILIZER | 0.1-1.0 |
| FOAM STABILIZER | 0.5-15 |
| THICKENING AGENT | 0.1-35 |
| HUMECTANTS | 5-30 |

Table 5, Table 6, and Table 7 summarize the stability results in percentage over the initial concentration of hydrogen peroxide and bleaching efficacy of the example bleaching gel at 50° C. (122° F.) of incubation for up to 1.5 months (M), 37° C. (98.6° F.) for up to 6 M and room temperature at 23° C. (73° F.) for up to 1.5 year, respectively.

The results show that the examples of the bleaching gel are stable for at least 1 month at 50° C., for at least 3 months at 37° C. and at least 1 year at room temperature, as more than 90% of its original hydrogen peroxide concentration was maintained and the bleaching efficacy maintained at the same level as the original gel (at time 0). In addition, the examples maintained gelled state during the stability testing periods at different temperatures.

TABLE 5
Stability results of the bleaching example gels at 50° C.

| | |
|---|---|
| Example 1 | Example 2 |

| Time | HP | BE | HP | BE |
|---|---|---|---|---|
| (days) | (%) | (min) | (%) | (min) |
| 0 | 100 | 80 | 100 | 56 |
| 7 | 97.7 | 81 | 98.8 | 52 |
| 14 | 95.3 | 80 | 98.2 | 48 |
| 30 | 91.2 | 89 | 94.5 | 57 |
| 45 | 86.7 | 151 | 89.5 | 144 |

TABLE 6
Stability results of the bleaching example gels at 37° C.

| | |
|---|---|
| Example 1 | Example 2 |

| Time | HP | BE | HP | BE |
|---|---|---|---|---|
| (Months) | (%) | (min) | (%) | (min) |
| 0 | 100 | 89 | 100 | 61 |
| 1 | 96.9 | 92 | 97.5 | 49 |
| 2 | 95.0 | 90 | 94.7 | 42 |
| 4 | 86.1 | 111 | 91.1 | 80 |
| 6 | 75.9 | N.D. | 82.0 | 217 |

TABLE 7
Stability results of the bleaching example
gels at room temperature (23° C.).

| | |
|---|---|
| Example 1 | Example 2 |

| Time | HP | BE | HP | BE |
|---|---|---|---|---|
| (Months) | (%) | (min) | (%) | (min) |
| 0 | 100 | 89 | 100 | 56 |
| 3 | 98.5 | 100 | 99.9 | 35 |
| 6 | 95.9 | 96 | 98.8 | 76 |
| 12 | 94.7 | N.D. | 94.2 | 47 |
| 20 | 88.3 | 92 | 89.0 | 54 |
| 24 | 80.05 | 139 | 85.5 | 116 |

When the bleaching gel was mixed with the activating gel, hydrogen peroxide was decomposed to form free radicals that oxidize the colored organic molecules and inorganic compounds within the tooth structure into colorless hydroxyl (alcohol like) groups, resulting in whitening the teeth.

Example 3 and Example 4 are exemplary bleaching gels.

The activating gels comprised the ingredients as recited in Table 3 (Example 3 and Example 4 represent Gels 4 and 5, respectively). The gel was degassed and then filled in 1.2 ml syringes, and the syringes were placed in an oven at 50° C. (122° F.), 37° C. (98.6° F.), and at room temperature at 23° C. (73° C.). At a predetermined time point, the syringes were taken out and the formulations were evaluated in terms of viscosity, and activating efficiency (in vitro on a FD & C dye). The viscosity of the gel was measured with a rheometer (DV-III Ultra, Brookfield Engineering, Middleboro, Mass.). At time 0, viscosity of the activator gels at 50 RPM ranged from 2500 cp to 3000 cp for example 3 and 800 cp to 1300 cp for example 4, respectively. The viscosity of the activator gel after incubation or room temperature storage was expressed in % over the initial viscosity (at time 0). The activating efficiency (AE) of the gel was evaluated by the time consumed for color of the activating gel mixed with the control bleaching gel and dye solution to apparently fade (i.e., turn colorless), so the shorter the time, the stronger the AE of the activating gel. The result is not considered as significantly different if the time difference is with 5-20 minutes.

Tables 8, 9 and 10 summarize the stability results in gel viscosity of and AE of the examples of the activator gel at 50° C. of incubation for up to 1.5 months, 37° C. for up to 6 months and room temperature for up to over 2 years, respectively. The results show that the examples of the activator gel are stable for at least one (1) month at 50° C., for at least four (4) months at 37° C. and at least one (1) year (12 months) at room temperature, as both the gel viscosity (expressed as % over the initial viscosity of the gel [at time 0] and the AE maintains the same level as the original gels [at time 0]).

TABLE 8
Stability Results of the Example Activating Gels at 50° C.

| | |
|---|---|
| Example 3 | Example 4 |

| Time | Viscosity | AE | Viscosity | AE |
|---|---|---|---|---|
| (days) | (%) | (min) | (%) | (min) |
| 0 | 100 | 80 | 100 | 58 |
| 7 | 97.9 | 81 | 104 | 53 |
| 14 | 92.2 | 82 | 104.5 | 48 |
| 30 | 87.7 | 85 | 97.1 | 62 |
| 45 | 99.4 | 109 | 96.8 | 114 |

TABLE 9
Stability Results of the Example Activating Gels at 37° C.

| | |
|---|---|
| Example 3 | Example 4 |

| Time | Viscosity | AE | Viscosity | AE |
|---|---|---|---|---|
| (months) | (%) | (min) | (%) | (min) |
| 0 | 100 | 80 | 100 | 58 |
| 1 | 97.6 | 88 | 76.8 | 52 |
| 2 | 95.8 | 94 | 91.7 | 65 |
| 4 | 95.1 | 114 | 84.2 | 71 |
| 6 | N.D. | N.D. | 89.1 | 103 |

TABLE 10
Stability Results of the Example Activating
Gels at Room Temperature.

| | |
|---|---|
| Example 3 | Example 4 |

| Time | Viscosity | AE | Viscosity | AE |
|---|---|---|---|---|
| (months) | (%) | (min) | (%) | (min) |
| 0 | 100 | 80 | 100 | 47 |
| 3 | N.D. | 86 | 95.2 | 45 |
| 6 | N.D. | N.D. | 83.6 | 60 |
| 9 | 96.8 | 104 | 101.6 | 56 |
| 12 | 103.0 | 78 | 83.5 | N.D. |
| 18 | 121.5 | 96 | 84.6 | 52 |
| 24 | 168.4 | 125 | 105 | 63 |

It will be appreciated that variations of the above-disclosed and other features and functions, or alternatives thereof, may be desirably combined into many other different systems or applications. Also various presently unforeseen or unanticipated alternatives, modifications, variations or improvements therein may be subsequently made by those skilled in the art, which are also intended to be encompassed by the following claims. Unless specifically recited in a claim, steps or components of claims should not be implied or imported from the specification or any other claims as to any particular order, number, position, size, shape, angle, color or material.

All references cited herein are herein incorporated by reference in entirety.

We claim herein:

1. A multi-component dental bleaching system comprising: a dental bleaching composition comprising a dental bleaching gel and a dental activator gel, wherein the dental bleaching gel comprises at least one peroxide, at least one bleaching agent stabilizer, at least one thickening agent and at least one foam stabilizer, and wherein the dental activator gel comprises a transition metal complex, at least one complex stabilizer, and optionally a flavorant.

2. The multi-component dental bleaching system of claim 1, wherein the at least one peroxide is selected from the group consisting of hydrogen peroxide, carbamide peroxide, urea peroxide, perborate, peroxyacetic acid, and combinations thereof.

3. The multi-component dental bleaching system of claim 1, wherein the at least one bleaching agent stabilizer is selected from the group consisting of a chelator, a tin salt, an alkali stannate, a phosphate, a pyrophosphate, a phosphoric acid, and combinations thereof.

4. The multi-component dental bleaching system of claim 1, wherein the at least one thickening agent is selected from the group consisting of poly(ethylene oxide) (PEO), wherein said PEO has a molecular weight greater than about 10,000; polyvinyl oxide (PVP); a copolymer of PEO and polypropylene oxide (PPO); and combinations thereof.

5. The multi-component dental bleaching system of claim 1, wherein the bleaching gel further comprises a humectant selected from glycerin, liquid polyethylene glycol (PEG), liquid polypropylene glycol, propylene glycol and combinations thereof.

6. The multi-component dental bleaching system of claim 1, wherein the foam stabilizer comprises at least one non-ionic surfactant and at least on colloidal silica.

7. The multi-component dental bleaching system of claim 6, wherein the transition metal of said complex is selected from water-soluble manganese, cobalt, nickel, and copper salts complexed with EDTA salts.

8. The multi-component dental bleaching system of claim 7, wherein the complex comprises transition metal-gluconate salt.

9. The multi-component dental bleaching system of claim 1, wherein the complex stabilizer is selected from the group of non-ionic polymer surfactant, polyethylene glycol (PEG), wherein the PEG has molecular weight greater than about 2000 Da; polyvinyl alcohol; polyoxyethylene-polypropylene block copolymer; and PVP.

10. The multi-component dental bleaching system of claim 1, wherein the dental bleaching system is in the form of a kit that includes the bleaching gel in a first part and the dental activator gel in a second part.

11. The multi-component dental bleaching system of claim 10, wherein the kit includes at least one duel compartment syringe containing the dental bleaching gel in a first compartment and the dental activator gel in a second compartment.

12. The multi-component dental bleaching system of claim 1, wherein the dental bleaching gel is mixed with the dental activator gel in a ratio from about 1:1 to about 5:1.

13. The multi-component dental bleaching system of claim 1, wherein the dental bleaching gel is stable to both heat and foaming for up to 12 months at room temperature.

14. The multi-component dental bleaching system of claim 1, wherein the dental activator gel is stable in consistency and activating efficacy for up to 12 months at room temperature.

15. The multi-component dental bleaching system of claim 1, wherein the dental activator gel has a pH of from about 7.5 to about 8.9, and the multi-component bleaching system has a final pH of about 7.0 to about 8.5.

16. A method of bleaching a subject's teeth comprising: applying a multi-component dental bleaching system containing: a dental bleaching composition comprising a dental bleaching gel and a dental activator gel, wherein the dental bleaching gel comprises at least one peroxide, at least one bleaching agent stabilizer, at least one thickening agent and at least one foam stabilizer, and wherein the dental activator gel comprises a transition metal complex and at least one complex stabilizer; mixing the dental bleaching gel and the dental activator gel in a ratio from about 1:1 to 5:1; and applying the mixed system to at least a portion of the subject's teeth.

17. The method of claim 16, wherein the multi-component bleaching system is applied with a dual-compartment syringe.

18. The method of claim 16, wherein the multi-component bleaching system is contained in a kit comprising at least one duel compartment syringe containing the dental bleaching gel in a first compartment and the dental activator gel in a second compartment.

19. The method of claim 16, wherein the dental activator gel has a pH of from about 7.5 to about 8.9, and the multi-component dental bleaching system has a final pH of about 7.0 to about 8.5.

20. The method of claim 16, wherein the multi-component bleaching system comprises a flavorant and a humectant.
